(19) United States
(12) Patent Application Publication
(10) Pub. No.: US 2016/0030438 A1
(43) Pub. Date: Feb. 4, 2016

(54) ANTI-TUMORAL COMPOSITION COMPRISING A PI3KBETA INHIBITOR AND A RAF INHIBITOR, TO OVERCOME CANCER CELLS RESISTANCE

(71) Applicant: SANOFI, Paris (FR)

(72) Inventors: Hélène BONNEVAUX, Paris (FR); Carlos GARCIA-ECHEVERRIA, Paris (FR); Angela VIRONE-ODDOS, Paris (FR)

(21) Appl. No.: 14/775,370

(22) PCT Filed: Mar. 14, 2014
(86) PCT No.: PCT/EP2014/055116

§ 371 Date: Sep. 11, 2015

(30) Foreign Application Priority Data
Mar. 15, 2013 (EP) 13305313.2

(51) Int. Cl. A61K 31/5377; G01N 33/50; A61K 31/437

(52) U.S. Cl. CPC A61K 31/5377; A61K 31/437; G01N 33/5011

(57) ABSTRACT

The present invention concerns a combination of a PI3Kβ inhibitor with a RAF inhibitor for its use for the treatment of a patient resistant to at least one RAF inhibitor, a kit comprising the same, its pharmaceutical uses thereof and a method of monitoring the efficiency of said combination when administered to a patient.

The present invention concerns a combination of a PI3Kβ inhibitor with a RAF inhibitor for its use in the treatment of a patient resistant to at least one RAF inhibitor, its pharmaceutical uses thereof and a method of monitoring the efficiency of said combination when administered to a patient.

Phosphoinositide 3-kinases (PI3Ks) are signalling molecules involved in numerous cellular functions such as cell cycle, cell motility and apoptosis. PI3Ks are lipid kinases that produce second messenger molecules activating several target proteins including serine/threonine kinases like PDK1 and AKT (also known as PKB). PI3Ks are divided in three classes and class I comprises four different PI3Ks named PI3K alpha, PI3K beta (PI3Kβ), PI3K delta and PI3K gamma.

2-{2-[(2S)-2-methyl-2,3-dihydro-1H-indol-1-yl]-2-oxoethyl}-6-(morpholin-4-yl)pyrimidin-4(3H)-one (here-below compound (I)) is a selective inhibitor of the PI3Kβ isoform. After treatment with this compound, cancer cells with an activated PI3K/AKT pathway, as for example PTEN-deficient tumor cells (Phosphatase and TENsin homolog gene, also known as phosphatase and tensin homolog mutated in multiple advanced cancers 1 gene), typically respond via inhibition of phosphorylation of AKT as well as of AKT downstream effectors, inhibition of tumor cell proliferation and tumor cell death induction. Several studies show that PI3Kβ isoform is the PI3K isoform involved in the tumorigenicity of PTEN-deficient tumors (V. Certal et al., J. Med. Chem. 2012, 55, 4788-4805 and V. Certal et al., Bioorganics & Medicinal Chemistry Letters, 22, (2012) 6381-6384; V. Certal et al., J Med Chem. 57 (2014):903-20).

RAF kinases participate in the RAS-RAF-MEK-ERK signal transduction cascade, also referred to as the mitogen-activated protein kinase (MAPK) cascade. The three RAF kinase family members are A-RAF, B-RAF and C-RAF. Cancer cells treated with inhibitors of RAF kinase typically respond via inhibition of phosphorylation of MEK and of ERK, down-regulation of Cyclin D, induction of G1 arrest, and finally undergo apoptosis. Thus, RAFs have been targets of great interest for the development of cancer therapeutics.

1-Propanesulfonamide,N-[3-[[5-(4-chlorophenyl)-1H-pyrrolo[2,3-b]pyridin-3-yl]carbonyl]-2,4-difluorophenyl] (here-below compound (II)) is an inhibitor of RAF kinases. This compound (here-below compound (II)), also known as PLX-4032 or vemurafenib is an orally available small-molecule, developed for the treatment of cancers harboring activating BRAF mutations. More particularly, it has marked antitumor effects against melanoma cell lines with the BRAF V600E mutation but not against cells with wild-type BRAF. Melanomas with the BRAF V600E mutation represent more than 50% of melanomas. They constitutively activate the mitogen-activated protein kinase (MAPK) pathway, promoting cell proliferation and preventing apoptosis.

In a recent phase I trial of vemurafenib, 81% of patients with BRAF mutated melanoma experienced at least 30% tumor shrinkage by Response Evaluation Criteria in Solid Tumors (RECIST) with a complete response in two patients (Flaherty K T et al., N. Engl J Med 363:809-819, 2010).

However, despite such promising responses, resistance to vemurafenib has emerged.

Drug resistance is the major reason for failure in cancer chemotherapy. Resistance may be either pre-existent (intrinsic resistance), or induced by drugs (acquired resistance). The acquired resistance appears after a transient response to the treatment with generally a relapse. Genetic mechanisms of acquired resistance to targeted kinase inhibitors are typically mutations affecting the target kinase or alterations of other genes within the target signaling pathway that may compensate for or bypass target oncoprotein inhibition.

The intrinsic resistance of cancer cells to RAF inhibitors has been studied. It has been shown that although PTEN expression status did not predict for sensitivity to the growth inhibitory effects of vemurafenib, the PTEN deficient/BRAF mutant melanoma cell lines showed significant less apoptosis than the cell lines wherein PTEN is expressed (Kim H. T. Paraiso et al., Cancer Res. 2011; 71:2750-2760). The co-occurrence of mutated BRAF and silencing of PTEN expression is relatively common in human melanoma (about 20-30%).

However, several mechanisms are involved in the resistance to RAF inhibitors. Actually, some BRAF mutated/PTEN deficient melanoma cells lines are sensitive to RAF inhibitors and some others are unsensitive to RAF inhibitors.

The mechanisms responsible for the intrinsic or acquired resistance over vemurafenib are still under study.

The currently available data suggest reactivation of the MAPK pathway through the emergence of truncated hyperactive forms of BRAF, secondary mutations in NRAS (the neuroblastoma RAS viral oncogene homologue) or MEK. For example, an activating mutation at codon 1221 in the downstream kinase MEK1 that was absent in the corresponding pretreatment tumor has been identified. The MEK1^{C121S} mutation was shown to increase kinase activity and confer robust resistance to RAF inhibition in vitro (Nikhil Wagle et al., J ClinOncol 29:3085-3096, 2011). Although MAPK reactivation occurs in many recurrent cases, increased PI3K signaling occurs in others.

Moreover, it has been described that in melanomas resistant to RAF or MEK inhibitors, TORC1 (TOR complex 1) activity was maintained after treatment with RAF or MEK inhibitors, in some cases despite strong suppression of MAPK signaling. TORC1 inhibition in response to RAF or MEK inhibitors, as measured by decreased pS6 (ribosomal protein S6, also called when phosphorylated pS6), may effectively predict induction of cell death by RAF inhibitor in BRAF mutant melanoma cells.

In in vivo mouse models, the suppression of TORC1 activity after MAPK inhibition was necessary for apoptosis induction and tumor response. In paired biopsies obtained from patients with BRAF mutant melanoma before treatment and after initiation of a RAF inhibitor therapy, pS6 suppression predicted significantly improved progression-free survival (PFS). Such a change in pS6 could be monitored in real time by serial fine-needle aspiration biopsies, making quantification of pS6 a valuable biomarker to guide treatment in BRAF mutant melanoma (Ryan B. Corcoran et al., Sci. Transl. Med. 5:196ra98, 2013).

Therefore, there is a need for a cancer therapy, in particular a melanoma therapy, which overcomes resistance to the RAF inhibitors. There is also a need to provide a treatment of cancer such as melanoma that is more effective in inhibiting tumor cell proliferation and enhancing tumor cell apoptosis. There is also a need to minimize toxicity towards patients.

There is a particular need for RAF inhibitor therapy used in combination with other targeted therapy leading to more efficiency without substantially increasing, or even maintaining or decreasing, the dosages of RAF inhibitor generally used.

There is also a need to provide biomarkers to monitor the efficiency of cancer therapies.

It is an object of the present invention to provide a treatment for a patient having cancer cells resistant to at least one RAF inhibitor.

It is an object of the present invention to provide a treatment for a patient having cancer cells resistant to at least one RAF inhibitor, which overcomes the resistance to said at least one RAF inhibitor.

It is an object of the present invention to provide a combination for its use for the treatment of a patient having resistant cancer cells to at least one RAF inhibitor.

It is a further object of the invention to provide a kit to treat a patient having cancer cells resistant to at least one RAF inhibitor.

It is an object of the invention to provide a medicament to treat a patient having cancer cells resistant to at least one RAF inhibitor.

It is another object of the invention to provide a method of treatment for a patient having cancer cells resistant to at least one RAF inhibitor.

It is an object of the invention to provide a biomarker and a method of monitoring the efficiency of a treatment for patients having cancer cells resistant to at least one RAF inhibitor.

The present invention thus relates to a combination of a PI3Kβ inhibitor with a RAF inhibitor for its use for the treatment of a patient having resistant cancer cells to at least one RAF inhibitor.

The invention also relates to a kit comprising the above mentioned combination for its use as mentioned above, for simultaneous, separate or sequential administration.

The invention also relates to a pharmaceutical composition comprising the combination of the invention for its use in the treatment of a patient having resistant cancer cells to at least one RAF inhibitor.

The invention also relates to a method of treatment comprising administering the above mentioned combination to a patient having resistant cancer cells to at last one RAF inhibitor.

The invention also relates to a biomarker and to a method of monitoring using said biomarker to monitor the efficiency of the combination as mentioned above when administered to a patient having resistant cancer cells to at least one RAF inhibitor.

Surprisingly, the inventors discovered that the combination of a RAF inhibitor together with a PI3Kβ inhibitor overcomes the resistance to at least one RAF inhibitor of cancer cells, especially in RAF inhibitor resistant melanoma cells, such as human melanoma A2058 cell line unsensitive to at least one RAF inhibitor.

Even more surprising, the combination as defined above shows a synergistic effect on cell lines resistant to at least one RAF inhibitor.

In one embodiment, by synergistic effect, it is understood that the effect of the combination is greater than the expected additive effect of its individual components. More particularly, the synergistic effect may be determined by the Ray method design as described in R.Straetemans, (Biometrical Journal, 47, 2005, 299-308).

In another embodiment, by synergistic effect, it may also be understood that the effect of the combination is greater than the best effect of one of the two individual components.

In another embodiment, synergy may by defined according toT. H. CORBETT et al., in that a combination manifests therapeutic synergy if it is therapeutically superior to one or other of the constituents used at its optimum dose (T. H. CORBETT et al., Cancer Treatment Reports, 66, 1187 (1982)). According to this definition, to demonstrate the efficacy of a combination, it may be necessary to compare the maximum tolerated dose of the combination with the maximum tolerated dose of each of the separate constituents in the study in question. This efficacy may be quantified, for example by the calculation the log₁₀ cells killed or any other known method.

In one embodiment, synergy according to the invention may be obtained in respect of one of the following effects:
anti-proliferative activity; and/or
pro-apoptotic activity.

In one embodiment, enhanced effect may be obtained in respect of S6 phosphorylation inhibition. By “enhanced effect” or “enhanced inhibition” is meant that the inhibitory effect of the combination is greater than the best inhibitory effect of one of the two individual components.

In one embodiment, synergy according to the invention may be obtained in respect of one of the following effects:
inhibition of tumor growth (tumor stasis); and/or
partial tumor regression; and/or;
complete tumor regression.

This(these) effect(s) may be obtained in cell line sensitive to or resistant to at least one RAF inhibitor.

One of the advantages of the present invention is to provide a new treatment for patients with a tumor showing RAF inhibitor resistance for which the therapeutic possibilities are few.

Another advantage of the invention is that thanks to the synergistic effect of the combination as above, lower doses of each active principle may be required to overcome resistance to RAF inhibitors and/or drugs toxicity may be reduced.

In one embodiment according to each object of the invention, PI3Kβ inhibitors are compounds which exhibit an inhibitory effect on the PI3Kβ. More particularly, they generally exhibit an inhibitory effect on PI3Kβ and moderate or no inhibitory effect on other PI3K isoforms, namely PI3Kalpha, PI3Kdelta and PI3Kgamma.

In one embodiment, they are selective towards PI3Kβ isoform. By “selective PI3Kβ inhibitor” it may be understood the ability of the PI3Kβ inhibitor to affect the particular PI3Kβ isoform, in preference to the other isoforms PI3Kalpha, PI3Kdelta and PI3Kgamma. The PI3Kβ selective inhibitors may have the ability to discriminate between these isoforms, and so affect essentially the PI3Kβ isoform. In one embodiment, the selective PI3Kβ inhibitors are not pan-PI3K inhibitors. This PI3Kβ isoform selectivity may exhibit better safety profiles compared to pan-PI3K inhibitors.

More particularly, in biochemical and cellular assays, selective PI3Kβ inhibitors may target PI3Kβ isoform with an IC₅₀≦300 nM and may be selective versus other PI3K isoforms, PI3K alpha, PI3K delta and PI3K gamma, with an IC₅₀≧250 nM. In one embodiment, they may exhibit a ratio of inhibition of PI3Kβ versus the others isoforms of at least 2 fold.

In one embodiment, said PI3Kβ inhibitors do not inhibit mTOR.

In one embodiment, the PI3Kβ inhibitor has the structural formula (I) as defined below:

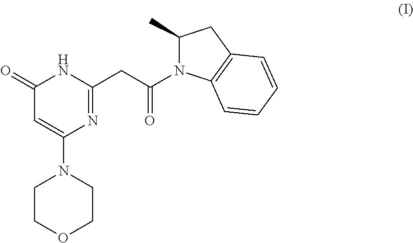 [SMILES: C[C@H]1Cc2ccccc2N1C(=O)Cc1nc(N2CCOCC2)cc(=O)[nH]1]

The PI3Kβ inhibitor according to formula (I) is referred to herein as “compound (I)” The compound (I) is a selective inhibitor of the PI3Kbeta isoform of the class I PI3K. By “selective inhibitor” it may be understood the ability of the compound (I) to affect the particular PI3Kβ isoform, in preference to the other isoforms PI3Kalpha, PI3Kdelta and PI3Kgamma. The compound (I) may have the ability to discriminate between, and so affect only the PI3Kβ isoform. More particularly, the compound (I) may have an inhibitory activity on the PI3Kβ isoform ten times superior to its inhibitory activity on the other isoforms alpha, delta and gamma.

The compound (I) may target PI3Kβ isoform with an IC₅₀ of 65 nM and may be selective versus other PI3K isoforms with an IC₅₀ of 1188 nM, 465 nM and superior to 10 000 nM on PI3Kalpha, PI3Kdelta and PI3Kgamma respectively, in biochemical assays.

The compound (I) may not inhibit mTOR, more particularly may not inhibit mTOR up to 10 μM.

Its selectivity was also controlled by profiling the compound (I) against a large panel of lipid and protein kinases comprising more than 400 kinases. Except PI3Kdelta and PI3Kβ isoform, VPS34 lipid kinase is the only kinase showing an inhibition with a submicromolar IC50 of 180 nM; nevertheless, this level of biochemical activity on VPS34 does not translate in cellular activity using a functional VPS34 cellular assay (1050 superior to 10,000 nM).

The high level of PI3Kβ-isoform selectivity observed in biochemical settings was confirmed in cellular assays.

In order to specifically explore compound of formula (I) cellular selectivity against each class I PI3K isoform separately, the inhibition of AKT phosphorylation on serine 473 residue (pAkt-S473) was evaluated in appropriate cellular systems (PIK3CA-mutated H460 lung tumor cells for PI3Kalpha, MEF-3T3-myr p110β mouse fibroblasts overexpressing activated p110β for PI3Kβ, MEF-3T3-myr p110δ mouse fibroblasts overexpressing activated p110δ for PI3Kdelta and RAW 264.7 mouse macrophages (after stimulation of AKT phosphorylation by C5a) for PI3Kgamma), as already described (Certal V, Halley F, Virone-Oddos A, Delorme C, Karlsson A, Rak A et al. Discovery and Optimization of New Benzimidazole- and Benzoxazole-Pyrimidone Selective PI3Kβ Inhibitors for the Treatment of Phosphatase and TENsin homologue (PTEN)-Deficient Cancers J. Med. Chem. 2012; 55:4788-4805).

The compound of formula (I) may inhibit PI3Kβ isoform in the PI3Kβ-dependent cell line with a potency 26-fold higher (IC50 of 32 nM) than on PI3Kdelta (IC50 of 823 nM).

The compound of formula (I) may exhibit the same level of activity on PI3Kalpha and PI3Kgamma isoform in cellular and biochemical assays (IC50s of 2,825 and >3,000 nM, respectively).

The compound of formula (I) may be a PI3Kβ-selective inhibitor in cells. The compound of formula (I) may be 26-fold, 88-fold and superior to 94-fold more potent on PI3Kβ than on PI3Kdelta, PI3Kalpha and PI3Kgamma, respectively.

The preparation, properties, and PI3Kβ-inhibiting abilities of compound (I) are provided in, for example, International Patent Publication No. WO2011/001114, particularly Example 117 and Table p 216 therein. The entire contents of WO2011/001114 are incorporated herein by reference. Neutral and salt forms of the compound of Formula (I) are all considered herein.

In one embodiment according to each object of the invention, RAF inhibitors are compounds which exhibit an inhibitory effect on the RAF proteins. More particularly, they generally exhibit an IC₅₀ towards RAF protein in a biochemical assay and in cells of less than 500 nM.

More specifically, RAF inhibitors are BRAF inhibitors. The following compounds can be cited as BRAF inhibitors: Sorafenib (Nexavar), Vemurafenib (PLX-4032), Dabrafenib (GSK2118436), PLX-4720, GDC-0879, Regorafenib (BAY 73-4506), RAF265 (CHIR-265), SB590885, AZ628, ZM 336372, NVP-BHG712, Raf265 derivative and GSK2118436.

In one embodiment, the RAF inhibitor has the structural formula (II) as defined below:

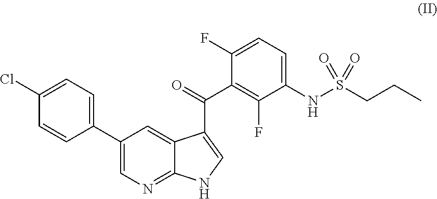 [SMILES: CCCS(=O)(=O)Nc1ccc(F)c(C(=O)c2c[nH]c3ncc(-c4ccc(Cl)cc4)cc23)c1F]

The RAF inhibitor according to formula (II) is referred to herein as “compound (II)” and is also known as PLX-4032 or Vemurafenib.

The preparation, properties, and RAF inhibiting abilities of compound (II) are provided in, for example, International Patent Publication No. WO 2007/002325, particularly Example 44 compound P-0956 and Tables 2a, 2b, 2c, 2d, 2e and 2h therein. The entire contents of WO2007/002325 are incorporated herein by reference. Neutral and salt forms of the compound of Formula (II) are all considered herein.

In some embodiments, the compounds described above could be unsolvated or in solvated forms. As known in the art, the solvate can be any of pharmaceutically acceptable solvent, such as water, ethanol, and the like. In general, the presence of a solvate or lack thereof does not have a substantial effect on the efficacy of the RAF or PI3Kβ inhibitor described above.

In some embodiments, these compounds are used in a pharmaceutically acceptable salt form. The salt can be obtained by any of the methods well known in the art, such as any of the methods and salt forms elaborated upon in WO 2011/001114, as incorporated by reference herein.

A “pharmaceutically acceptable salt” of the compound refers to a salt that is pharmaceutically acceptable and that retains pharmacological activity. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, or S. M. Berge, et al., “Pharmaceutical Salts,” J. Pharm. Sci., 1977; 66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, as well as those salts formed with organic acids, such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid.

In one embodiment, the combination for its use according to the invention, can either inhibit tumor cells growth, or achieve partial or complete tumor cells regression.

According to an embodiment, the present invention relates to the combination for its use as defined above, wherein the PI3Kβ inhibitor and the RAF inhibitor are in amounts that produce a synergistic effect, as defined above.

In one embodiment, the combination for its use according to the invention enhances anti-proliferative activity and pro-apoptotic activity on cancer cells of the patient.

According to an embodiment, the present invention relates to the combination for its use as defined above, wherein the PI3Kβ inhibitor and the RAF inhibitor are in amounts that produce a synergistic effect and/or a stimulatory effect on the antiproliferative activity and on the pro-apoptotic activity on cancer cells of the patient. In one embodiment, said synergistic effect and/or a stimulatory effect on the pro-apoptotic activity on cancer cells of the patient is obtained in a concentration-dependent manner.

In a particular embodiment, said synergistic effect on the anti-proliferative activity may be reached for a ratio compound (I)/compound (II) comprised from 1/16 to 26/1.

In a particular embodiment, said stimulatory effect on the pro-apoptotic activity may be reached for the concentrations of 1 μM to 10 μM of compound (II) combined with compound (I) at a concentration of 10 μM.

In a particular embodiment, said stimulatory effect on the pro-apoptotic activity may be reached for the concentrations of 10 μM or 1 μM of compound (II) combined with compound (I) at a concentration of 10 μM.

In a particular embodiment, said stimulatory effect on the pro-apoptotic activity may be reached for the concentrations of 0.1 μM to 10 μM of compound (II) combined with compound (I) at a concentration of 0.1 μM to 10 μM.

In a particular embodiment, said stimulatory effect on the pro-apoptotic activity may be reached for the concentrations of 0.1 μM, 1 μM or 10 μM of compound (II) with 0.1 μM, 1 μM or 10 μM of compound (I).

In a particular embodiment, said stimulatory effect on the pro-apoptotic activity may be reached for the concentrations of 10 μM of compound (II) with 10 μM of compound (I).

In a particular embodiment, said stimulatory effect on the pro-apoptotic activity may be reached for the concentrations of 10 μM of compound (II) with 1 μM of compound (I).

In a particular embodiment, said stimulatory effect on the pro-apoptotic activity may be reached for the concentrations of 1 μM of compound (II) with 10 μM of compound (I).

In a particular embodiment, said stimulatory effect on the pro-apoptotic activity may be reached for the concentrations of 1 μM of compound (II) with 1 μM of compound (I).

In a particular embodiment, said stimulatory effect on the pro-apoptotic activity may be reached for the concentrations of 0.1 μM of compound (II) with 10 μM of compound (I).

In a particular embodiment, said inhibitory effect on the S6 phosphorylation may be reached for the concentrations of 0.1 μM to 10 μM of compound (II) combined with compound (I) at a concentration of 0.1 μM to 10 μM.

In a particular embodiment, said inhibitory effect on the S6 phosphorylation may be reached for the concentrations of 0.1 μM, 1 μM or 10 μM of compound (II) with 0.1 μM, 1 μM or 10 μM of compound (I).

In one embodiment of each object of the invention, the patient resistant to the at least one RAF inhibitor is resistant to said RAF inhibitor of the combination. In one embodiment, the resistance to the at least one RAF inhibitor is an intrinsic resistance. In one embodiment, the resistance to the at least one RAF inhibitor is an acquired resistance.

In one embodiment, when the resistance as defined above is an acquired resistance, it is to be understood that the patient resistant to at least one RAF inhibitor has been previously treated by said RAF inhibitor and does not respond anymore to the treatment or could respond to the treatment with high and too toxic doses of said RAF inhibitor.

In one embodiment, the resistant cancer cells of the patient are relatively resistant to the RAF inhibitor. In one embodiment, the resistant cancer cells of the patient are unsensitive cancer cells to the RAF inhibitor. By “unsensitive”, it may be understood that the cancer cells do not respond to the RAF inhibitor at pharmaceutically acceptable doses. More particularly, the RAF inhibitor may show an IC50 at least ten times superior with unsensitive cancer cells than with sensitive cancer cells.

For example, the Vemurafenib BRAF inhibitor may show approximately an 1050 of 4,200 nM with A2058 unsensitive cancer cells and an IC50 of 84 nM with WM-266-4 sensitive cancer cells.

In one embodiment, the cancer cells present an activating BRAF mutation, particularly a BRAF-V600E mutation or a BRAF-V600K mutation.

Various activating mutations (ie, somatic point mutations) in BRAF cause the protein to become overactive. This triggers a signaling cascade that can play a role in specific malignancies. Approximately 90% of known BRAF mutations are V600E mutations. These involve the substitution of glutamic acid (E) for valine (V) at position V600 of the protein chain, resulting in constitutively active BRAF. Other variants of this point mutation include lysine (K), aspartic acid (D), and arginine (R). The V600 point mutation allows BRAF to signal independently of upstream cues. As a result of constitutively active BRAF, overactive downstream signaling via MEK and ERK leads to excessive cell proliferation and survival, independent of growth factors.

By “activating BRAF mutation”, it may be understood a mutation on the gene BRAF which allows BRAF to signal independently of upstream cues and/or which produces a constitutively active BRAF protein.

In another embodiment, the cancer cells are PTEN deficient. The treated cancer can therefore be a BRAF-mutated, such as a BRAF-V600E mutated/PTEN deficient melanoma or a BRAF-V600K mutated/PTEN deficient melanoma.

Cancers to be treated according to the present invention are chosen from the group consisting of: breast cancer, lung cancer, colon cancer, thyroid cancer, endometrium and ovarian cancers and melanomas. In a particular embodiment, the cancer is a melanoma.

In a particular embodiment, the patient to be treated has a mutated MEK1 kinase, more particularly a mutated MEK1^{C121S} kinase.

In one embodiment according to each object of the invention, a PI3Kβ inhibitor and a RAF inhibitor are in a combined preparation for simultaneous, separate or sequential administration for use in the treatment of a patient having resistant cancer cells to at least one RAF inhibitor.

According to the invention, “simultaneous” means that the PI3Kβ inhibitor and the RAF inhibitor are administered by the same route and at the same time (eg they can be mixed), “separate” means they are administered by different routes and/or at different times, and “sequential” means they are administered separately, at different times.

Simultaneous administration typically means that both compounds enter the patient at precisely the same time. However, simultaneous administration also includes the possibility that the RAF inhibitor and PI3Kβ inhibitor enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds.

In other embodiments, the RAF and PI3Kβ inhibitors are not simultaneously administered. In this regard, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient.

In a particular embodiment, the administration is separate or sequential and the administration of the PI3Kβ inhibitor is followed by the administration of the RAF inhibitor.

In another particular embodiment, the administration is separate or sequential and the administration of the RAF inhibitor is followed by the administration of the PI3Kβ inhibitor.

In another embodiment, the combined preparation as mentioned above is comprised in a kit, further comprising instructions for use.

According to each object of the invention, in one embodiment:
the compound (I), is administered at a dose comprised from 100 to 1600 mg, and
the compound (II), is administered at a dose comprised from 600 to 1100 mg.

More particularly:
the compound (I), is administered at a dose selected from the following doses: 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, and 1600 mg, typically selected from the following doses:100, 200, 400, 600, 800, 1000, 1200, 1400 and 1600 mg, and
the compound (II), is administered at a dose of 720 mg or 960 mg, typically 960 mg.

In a further embodiment, according to each object of the invention, the compounds (I) and (II) are administered twice a day. In one embodiment, the compounds (I) and (II) are administered orally. The cycle of administration generally lasts at least 28 days, typically 28 days. The cycle of administration can be repeated, with or without period of rest (i.e. period without administration of the compounds (I) and (II)) between two cycles. More particularly, the compounds (I) and (II) are administered for a period of at least 28 days without rest.

“Dose” means the administration dose (for example in the expression “Vemurafenib is administered at a dose from 600 to 1100 mg.”). The dose is not necessarily the “unit dose”, i.e. a single dose which is capable of being administered to a patient, and which can be readily handled and packaged, remaining as a physically and chemically stable unit dose. As an example, usually, when the dose of Vemurafenib is 960 mg (administration dose), 4 tablets of 240 mg (unit dose) may be administered.

When the compound or product is administered twice daily, the dose per day is twice the administration dose (i.e. the dose in the present application). For example, when a dose of 960 mg of Vemurafenib is administered twice a day, the total daily dose is 1,920 mg.

In one embodiment, the combination and/or the kit and/or the medicament for their use as mentioned abovecomprise(s) at least one further anticancer compound.

In one embodiment, the combination and/or the kit and/or the pharmaceutical composition for their use as mentioned abovefurther comprise(s) at least one pharmaceutically acceptable excipient.

In one embodiment, the invention relates to the use of a combination as mentioned above for the preparation of a medicament to treat patients having cancer cells resistant to at least one RAF inhibitor.

In another aspect, the invention relates to methods of treating a patient with cancer cells resistant to at least one RAF inhibitor that comprise administering to the patient a therapeutically effective amount of a PI3Kβ inhibitor, in combination with a RAF inhibitor.

In cancer cells resistant to RAF inhibitors, the level of pS6 may not be decreased, indicating that TORC1 activity is not suppressed. Thus, the inhibition of the phosphorylation of S6 may be used as a biomarker to monitor the beneficial activity of the combination as defined above:

if the phosphorylation of S6 is inhibited after administration of the combination as defined above, meaning that the level of pS6 in the resistant cancer cells decreases, it shows that the resistance may be overcome by the combination as defined above.

By “pS6” is meant the phosphorylated ribosomal protein S6.

Thus, in another aspect, the invention relates to the use of protein pS6 as a biomarker of the efficiency of a combination comprising a PI3Kβ inhibitor and a RAF inhibitor on cancer cells resistant to at least one RAF inhibitor. In a particular embodiment, said combination is the combination according to the invention.

In one embodiment, the invention relates to an in vitro method of monitoring the response of a patient, having cancer cells resistant to at least one RAF inhibitor, to the combination as defined above, said method comprising:
i) determining the amount of protein pS6 in cancer cells resistant to at least one RAF inhibitor of said patient at a first time point,
ii) determining the amount of protein pS6 in cancer cells resistant to at least one RAF inhibitor of said patient at a later time point,
iii) comparing the amount of protein pS6 of step i) with the amount of protein pS6 in step ii), and
iv) determining that the patient responds to said combination if the amount of protein pS6 of step i) is equal or superior to the amount of protein pS6 in step ii).

By “the patient responds” is meant that the combination of the invention reduces or suppresses TORC1 activity, leading to a stabilization or to a decrease of the phosphorylation of S6 level, and in particular to a stabilization or a decrease of the disease.

In one embodiment, in step iv), the amount of protein pS6 of step i) is superior to the amount of protein pS6 in step ii). More particularly, the amount of protein pS6 of step i) is superior by at least 30% of the amount of protein pS6 in step ii), preferably by at least 50% of the amount of protein pS6 in step ii).

In one embodiment, the step i) is performed before the administration of said combination and the step ii) is performed after the administration of said combination to the patient. In this particular embodiment, it may be determined in step iv) that the patient responds to said combination if the amount of protein pS6 of step i) is superior to the amount of protein pS6 in step ii).

In another embodiment, steps i) and ii) are both performed after administration to the patient of said combination, at different time points. In this particular embodiment, it may be determined in step iv) that the patient responds to said combination if the amount of protein pS6 of step i) is equal or superior to the amount of protein pS6 in step ii).

In one embodiment, the amount of protein pS6 could be determined by western blotting. Other methodologies could be used to monitor the level of pS6 inhibition.

In one embodiment, the resistant cancer cells of the patient are unsensitive cancer cells to at least one RAF inhibitor.

In general, the PI3Kβ and RAF inhibiting compounds, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, more particularly in unit dosage forms suitable for simple administration of precise dosages. A particular route of administration is oral, particularly one in which a convenient daily dosage regimen can be adjusted according to the degree of severity of the disease to be treated.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitanmonolaurate, triethanolamineoleate, butylatedhydroxytoluene, and the like.

Dosage forms suitable for parenteral injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms also may comprise buffering agents.

Solid dosage forms as described above can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., RAF or PI3Kβ inhibitor compound described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylatedisostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds described herein with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration may include, for example, ointments, powders, sprays, and inhalants. The active component is admixed under sterile conditions with a physiologically acceptable carrier and any preservatives, buffers, or propellants as can be required. Ophthalmic formulations, eye ointments, powders, and solutions also can be employed.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compounds described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

According to the invention, each of the embodiments can be taken individually or in all possible combinations.

Examples have been set forth below for the purpose of illustration and to describe certain specific embodiments of the invention. However, the scope of the claims is not to be in any way limited by the examples set forth herein.

### BRIEF DESCRIPTION OF THE DRAWINGS

FIG. 1 is an isobologram representation of the in vitro anti-proliferative activity of compound (I) in combination with compound (II) in human melanoma cell line WM-266-4.

FIG. 2 is an isobologram representation of the in vitro anti-proliferative activity of compound (I) in combination with compound (II) in human melanoma cell line A2058.

### EXAMPLES

Several in vitro experiments have been conducted in order to study the interaction between a PI3Kβ inhibitor (compound I) and a BRAF inhibitor (compound II) on the inhibitory activity on cell proliferation, on the induction of cell death and on S6 phosphorylation in human melanoma cell lines WM-266-4 and A2058 (both BRAF mutant and PTEN deficient). Melanoma cell line A2058 has been shown to be unsensitive to compound (II) as single agent with a lower antiproliferative effect compared to sensitive cell line as WM-266-4 (approximately IC40 of 1,200 nM and 60 nM respectively).

The interaction between compound (I) and compound (II) on both cell lines was characterized using ray design approach as described in R.Straetemans, (Biometrical Journal, 47, 2005) which allows to investigate synergy for different effective fraction f of the compounds in the mixture, the effective fraction being constant for each ray. Representative experiments for each combination and each cell line are presented hereunder. The cell death induced by both compounds alone or in combination was characterized using western blotting method which allows investigating apoptosis by detecting the cleavage of the PARP protein.

The inhibitory effect on ribosomal S6 protein phosphorylation by both compounds alone or in combination was characterized using western blotting method which allows investigating S6 phosphorylation by detecting the expression of ribosomal protein S6 phosphorylated on Ser240/244 position (pS6).

### Example 1

### In Vitro Anti-Proliferative Activity of Compound (I) in Combination with Compound (II) in Human Melanoma Cell Line WM-266-4

To evaluate the anti-proliferative activity of the PI3Kβ selective inhibitor compound (I) in combination with the BRAF inhibitor compound (II), experiments were conducted using human melanoma cell line WM-266-4 (BRAF mutant and PTEN-deficient). Prior to in vitro combination studies, the activity of individual agents was investigated using WM-266-4 cell line. The purpose of testing individual agents was to determine the independence of their action and to determine the dilution design of the Fixed Ratio Drug Combination assay. The characterization of the interaction between compound (I) and compound (II) was studied using the ray design method and associated statistical analysis, which evaluates the benefit of the combination at different drug efficacy ratios.

Material and Methods

The human melanoma WM-266-4 cell line was purchased at ATCC (Ref number CRL-1676 Batch 3272826). The WM-266-4 cells were cultured in RPMI1640 medium supplemented with 10% FBS and 2 mM L-Glutamine.

Compound (I) and compound (II) were dissolved in DMSO at concentration of 30 mM. They were diluted serially, in DMSO following a 3 or 3.3-fold dilution step in order to obtain 10 mM to 0.03 μM solutions: then each solution was diluted 50-fold in culture medium containing 10% serum before being added onto cells with a 20-fold dilution factor. The final concentrations tested were defined by a Ray design which allows characterizing the interaction of the two compounds for several fixed proportions in the mixture. The ray design used for this experiment includes one ray for each single agent and 4 combination rays. All rays have 10 concentrations (see Table 1). The DMSO concentration was 0.1% in controls and in all treated wells.

TABLE 1
Ray Design of Example 1
Table 1 provides the ray design used to perform the example 1 study.
Concentrations are given in nM.
Ray 1: Compound (I) alone

| | | | | | | | | | | |
|---|---|---|---|---|---|---|---|---|---|---|
| (I) | 30000 | 10000 | 3000 | 1000 | 300 | 100 | 30 | 10 | 3 | 1 |
| (II) | 0 | 0 | 0 | 0 | 0 | 0 | 0 | 0 | 0 | 0 |

Ray 2

| | | | | | | | | | | |
|---|---|---|---|---|---|---|---|---|---|---|
| (I) | 30000 | 10000 | 3000 | 1000 | 300 | 100 | 30 | 10 | 3 | 1 |
| (II) | 3000 | 1000 | 300 | 100 | 30 | 10 | 3 | 1 | 0.0 | 0.1 |

Ray 3

| | | | | | | | | | | |
|---|---|---|---|---|---|---|---|---|---|---|
| (I) | 30000 | 10000 | 3000 | 1000 | 300 | 100 | 30 | 10 | 3 | 1 |
| (II) | 1000 | 300 | 100 | 30 | 10 | 3 | 1 | 0.3 | 0.1 | 0.03 |

Ray 4

| | | | | | | | | | | |
|---|---|---|---|---|---|---|---|---|---|---|
| (I) | 30000 | 10000 | 3000 | 1000 | 300 | 100 | 30 | 10 | 3 | 1 |
| (II) | 300 | 100 | 30 | 10 | 3 | 1 | 0.3 | 0.1 | 0.03 | 0.01 |

Ray 4 bis

| | | | | | | | | | | |
|---|---|---|---|---|---|---|---|---|---|---|
| (I) | 30000 | 10000 | 3000 | 1000 | 300 | 100 | 30 | 10 | 3 | 1 |
| (II) | 100 | 30 | 10 | 3 | 1 | 0.3 | 0.1 | 0.03 | 0.01 | 0.003 |

Ray 5: Compound (II)alone

| | | | | | | | | | | |
|---|---|---|---|---|---|---|---|---|---|---|
| (I) | 0 | 0 | 0 | 0 | 0 | 0 | 0 | 0 | 0 | 0 |
| (II) | 10000 | 3000 | 1000 | 300 | 100 | 30 | 10 | 3 | 1 | 0.3 |

WM-266-4 cells were plated at 2500 cells/well in 96-well plates in appropriate culture medium and incubated for 6 hours at 37° C., 5% CO₂. Cells were treated in a grid manner with increasing concentrations of compound (I) ranging from 1 to 30,000 nM and with increasing concentrations of compound (II) ranging from 0.001 to 10,000 nM, depending on the given drug ratio, and incubated for 96 hours. Cell growth was evaluated by measuring intracellular ATP using CelltiterGlo® reagent (Promega) according to the manufacturer's protocol. Briefly, CellTiterGlo® was added to each plate, incubated for 1 hour then luminescent signal was read on the MicroBeta Luminescent plate reader. Three experiments have been performed on the cell line. For each experiment, two 96-well plates are used allowing working with duplicates.

Inhibition of cell growth was estimated after treatment with one compound or the combination of compounds for four days and comparing the signal to cells treated with vehicle (DMSO).

Growth inhibition percentage (GI %) was calculated according to the following equation:

GI %=100*(1−((X−BG)/(TC−BG))

where the values are defined as:

X=Value of wells containing cells in the presence of compounds (I) or (II) alone or in combination

BG=Value of wells with medium and without cells

TC=value of wells containing cells in the presence of vehicle (DMSO).

From the growth inhibition percentage, absolute IC40 is defined as the concentration of compound where GI % is equal to 40%.

This measurement allows determining the potential synergistic combinations using the statistical method described hereunder.

The relative potency ρ is first estimated as

$$ρ = \frac{IC {40}_{( 1 )}}{IC {40}_{( 2 )}}$$

where IC40₍₁₎ is the IC40 of the compound (I) and IC40₍₂₎ is the IC40 of the compound (II).

This effective fraction for the ray i is then calculated as

$${f}_{i} = \frac{1}{{c}_{i} \cdot ρ + 1} where {c}_{i} = \frac{[ ( 2 ) ]}{[ ( 1 ) ]}$$

is the constant ratio of the concentrations of the compounds (I) and (II) in the mixture.

A global non linear model using NLMIXED procedure of the software SAS V9.2 was applied to fit simultaneously the concentration-responses curves for each ray. The model used is a4-parameter logistic model corresponding to the following equation:

$${Y}_{ikj} = E {min}_{i} + \frac{( E {max}_{i} - E {min}_{i} )}{1 + exp [ - {m}_{i} log ( \frac{{Conc}_{ij}}{IC {50}_{i}} ) ]} + {ɛ}_{ijk}$$

Yᵢⱼₖ is the percentage of inhibition for the k^{th} replicate of the j^{th} concentration in the i^{th} ray Concᵢⱼ is the j^{th} mixture concentration (sum of the concentrations of compound (I) and compound (II)) in the i^{th} ray

Eminᵢ is the minimum effect obtained from i^{th} ray

Emaxᵢ is the maximum effect obtained from i^{th} ray

IC50ᵢ is the IC50 obtained from i^{th} ray

mᵢ is the slope of the curve adjusted with data from i^{th} ray

εᵢⱼₖ is the residual for the k^{th} replicate of the j^{th} concentration in the i^{th} ray, εᵢⱼₖ˜N(0, σ²)

Emin, Emax and/or slope were shared whenever it was possible without degrading the quality of the fit.

The combination index Ki of each ray and its 95% confidence interval was then estimated using the following equation based on the Loewe additivity model:

$$\frac{{C}_{( 1 )}}{IC {40}_{( 1 )}} + \frac{{C}_{( 2 )}}{IC {40}_{( 2 )}} = {K}_{i}$$

where IC40₍₁₎ and IC40₍₂₎ are the concentrations of compound (I) and compound (II) necessary to obtain 40% of inhibition for each compound alone and C₍₁₎ and C₍₂₎ are the concentrations of compound (I) and compound (II) in the mixture necessary to obtain 40% of inhibition.

Additivity was then concluded when the confidence interval of the combination index (Ki) includes 1, significant synergy was concluded when the upper bound of the confidence interval of Ki is less than 1 and significant antagonism was concluded when the lower bound of the confidence interval of Ki is higher than 1.

The isobologram representation permits to visualize the position of each ray according to the additivity situation represented by the line joining the point (0,1) to the point (1,0). All rays below this line correspond to a potential synergistic situation whereas all rays above the line correspond to a potential antagonistic situation.

Results of In Vitro Studies

Compound (I), as single agent, inhibited the proliferation of WM-266-4 cells with an IC40 of 6,688 nM. Compound (II), as single agent, inhibited the proliferation of WM-266-4 cells with an IC40 of 35 nM (see table 2 below).

TABLE 2
Absolute IC₄₀ estimations for
each compound alone in example 1
Absolute IC₄₀ of single agents are esti-
mated with a 4-parameter logistic model

| | Absolute IC40s (nM) | |
|---|---|---|
| Compound (I) | 6,687 | [1,809; 11,566] |
| Compound (II) | 34.9 | [28.6; 41.3] |

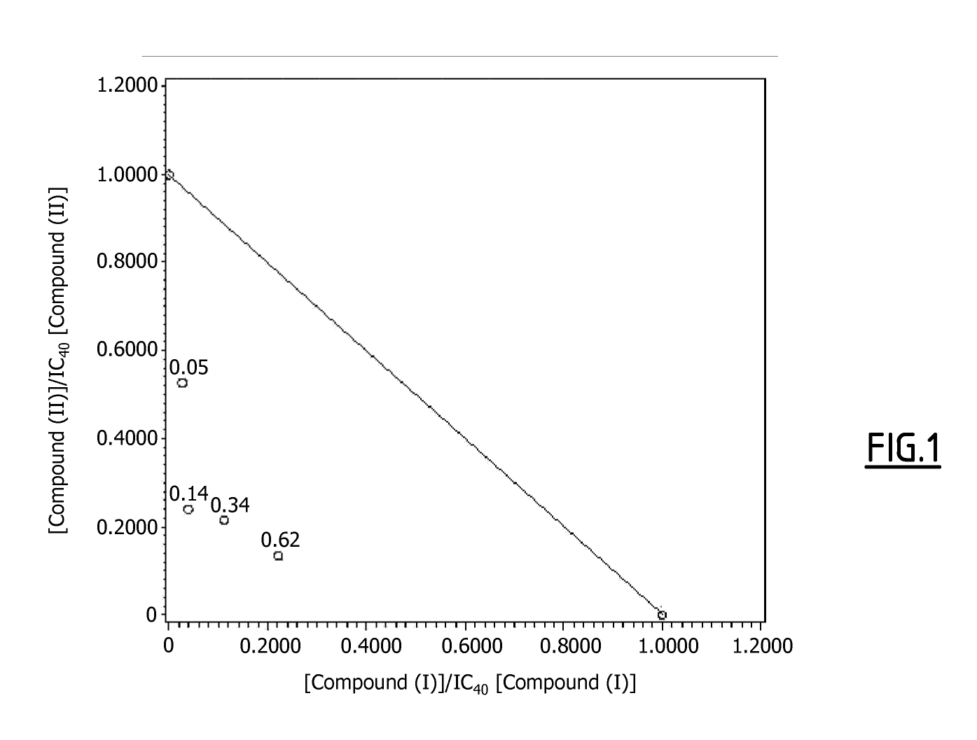

From the isobologram representation (FIG. 1) and the Table 3, significant synergy is observed with a Ki ranging from 0.28 to 0.55 for effective fraction f of compound (I) in the mixture between 0.05 and 0.62 which correspond to the situation where compound (I) is equally or less present than compound (II) in the mixture.

TABLE 3
Interaction characterization in example 1
Interaction indexes (Ki) allow us to define the
interaction observed between the two compounds.

| | | Ki (confidence | | Interaction |
|---|---|---|---|---|
| | f values | interval at 95%) | | characterization |
| Ray 2 | 0.05 | 0.5545 | [0.4155; 0.6936] | Synergy |
| Ray 3 | 0.14 | 0.2795 | [0.1923; 0.3668] | Synergy |
| Ray 4 | 0.34 | 0.3279 | [0.2033; 0.4526] | Synergy |
| Ray 4bis | 0.62 | 0.3562 | [0.1693; 0.5431] | Synergy |

These data correspond to a representative study out of 3 independent experiments. For these three experiments, synergy or additivity with tendency to synergy was observed for an effective fraction f between 0.04 and 0.62.

### Example 2

### In Vitro Pro-Apoptotic Activity of Compound (I) in Combination with Compound (II) in Human Melanoma Cell Line WM-266-4

To evaluate the pro-apoptotic activity of the PI3Kβ selective inhibitor compound (I) in combination with the BRAF inhibitor compound (II), experiments were conducted using the human melanoma cell line WM-266-4 (BRAF mutant and PTEN-deficient). The characterization of the interaction between compound (I) and compound (II) was studied using western blotting method measuring the expression of cleaved PARP which allows to investigate apoptosis by detecting the cleavage of the PARP protein.

Material and Methods

The human melanoma WM-266-4 cell line was purchased at ATCC (Ref number CRL-1676 Batch 3272826). The WM-266-4 cells were cultured in RPMI1640 medium supplemented with 10% FBS and 2 mM L-Glutamine.

Compound (I) and compound (II) were dissolved in DMSO at concentration of 10 mM. They were diluted following a 10 fold step dilution in DMSO in order to obtain a 1 mM solution. Then each solution at 10 mM and 1 mM was diluted 50-fold in culture medium containing 10% serum before being added onto cells with a 20-fold dilution factor to reach final concentrations of 10,000 nM and 1,000 nM. The final DMSO concentration was 0.1% in controls and in all treated wells.

WM-266-4 cells were seeded into 6-well microplates at 1 000 000 cells per well, in complete culture medium and incubated at 37° C., 5% CO₂, overnight. Then, the cells were incubated in the presence or absence of compound (I) and in the presence or absence of compound (II) for 24 hours at 37° C. in the presence of 5% of CO₂.

At the end of cell treatment period, adherent cells as well as cells in the cell culture supernatant were lysed for the preparation of the proteins. Cells were lysed in a lysis buffer containing Hepes 50 mM, NaCl 150 mM, Glycerol 10%, Triton 1%, pH=7.5, adding extemporaneously a cocktail of protease and phosphatase inhibitors diluted 100 fold. Protein concentrations in each sample were determined using microBCA technique according to manufacturer's instructions. Western blotting was performed loading 20 μg of proteins in each gel well, and according to the operating procedure. PARP cleavage was revealed using cleaved PARP (asp214) rabbit polyclonal antibody followed by an anti-rabbit IgG HRP conjugate antibody. GAPDH was revealed in control using anti-GAPDH rabbit monoclonal antibody 14C10 followed by an anti-rabbit IgG HRP conjugate antibody. After western blotting revelation according to the operating procedure instructions, luminescence was read using FujiFilm (Ray Test) apparatus.

This instrument measures the total signal of luminescence obtained on Fujifilm machine (AU) for each selected band. Then, it subtracts the background value (BG) proportional to the size of the selected band or Area. The background is calculated from a band taken on the specific background of the western blot, to obtain the specific signal or (AU−BG) for each band. The calculation of the fold induction is performed for each treatment with compound or combination of compounds using the following formula:

Fold Induction=((AU−BG)t/(AU−BG)st)*100

Where the values are defined as:

(AU−BG)t=Value of wells containing cells treated with the compounds (I) or (II) alone or in combination

(AU−BG)st=value of wells containing cells treated with the solvent (DMSO).

Results of In Vitro Studies

As compared to untreated cells, compound (I) or compound (II) as single agent at 10,000 nM concentration did not induce any significant WM-266-4 cell apoptosis, with a fold induction of PARP cleavage of 0.71 and 1.38, respectively. In the combination arm where the cells were treated with 10,000 nM of compound (I) and 10,000 nM of compound (II), as compared to untreated cells, the combined treatment induced WM-266-4 cell apoptosis with a fold induction of PARP cleavage of 3.26. In the combination arm where the cells were treated with 10,000 nM of compound (I) and 1,000 nM of compound (II), as compared to untreated cells, combined treatment induced cell apoptosis with a fold induction of 4.31. Taxotere is shown as a positive control of apoptosis induction with a fold induction of PARP cleavage of 2.88 (see table 4).

These data correspond to a representative study out of 2 independent experiments. GAPDH expression has been controlled in the lower panel of the western blots as a loading control.

TABLE 4
WM-266-4 cell apoptosis induction for each compound
alone or in combination in example 2

| Concentration | PARP cleavage | Fold |
|---|---|---|
| (nM) | signal (AU-BG) | induction |
| DMSO 0.1% | 713424 | 1.0 |
| Compound (I) 10 000 nM | 509366 | 0.71 |
| Compound (II) 10 000 nM | 982219 | 1.38 |
| Compound (I) 10 000 nM | 2327032 | 3.26 |
| Compound (II) 10 000 nM | | |
| Compound (I) 10 000 nM | 3075666 | 4.31 |
| Compound (II) 1 000 nM | | |
| Taxotere 100 nM | 2054900 | 2.88 |

### Example 3

### In Vitro Anti-Proliferative Activity of Compound (I) in Combination with Compound (II) in Human Melanoma Cell Line A2058

To evaluate the anti-proliferative activity of the PI3Kβ selective inhibitor compound

(I) in combination with the BRAF inhibitor compound (II), experiments were conducted using human melanoma cell line A2058 (BRAF mutant, PTEN-deficient and unsensitive to said BRAF inhibitor). The characterization of the interaction between compound (I) and compound (II) was studied using the ray design method and associated statistical analysis, which evaluates the benefit of the combination at different drug efficacy ratios.

Material and Methods

The human melanoma A2058 cell line was purchased at ATCC (Ref number CRL-11147 Batch 5074651). The A2058 cells were cultured in DMEM High glucose medium supplemented with 10% FBS and 2 mM L-Glutamine.

Compounds (I) and (II) dilutions were prepared according to the material and methods of example 1. The final concentrations tested were defined by Ray design method described below. The DMSO concentration was 0.1% in controls and in all treated wells.

A ray design was used allowing the characterization of the interaction of the two compounds for several fixed proportion in the mixture. The ray design includes one ray for each single agent and 19 combination rays. The ray with compound (I) alone has 14 concentrations, the ray with compound (II) alone has 18 concentrations and the combination rays have between 7 and 14 concentrations.

A2058 cells were plated at 4,000 cells/well in 384-well plates in appropriate culture medium and incubated for 6 hours at 37° C., 5% CO₂. Cells were treated in a grid manner with increasing concentrations of compound (I) ranging from 0.01 to 30,000 nM and with increasing concentrations of compound (II) ranging from 0.0001 to 30,000 nM and incubated for 96 hours. Cell growth was evaluated by measuring intracellular ATP using CelltiterGlo® reagent (Promega) according to the manufacturer's protocol. Briefly, CellTiterGlo® was added to each plate, incubated for 1 hour then luminescent signal was read on the MicroBeta Luminescent plate reader.

Four experiments have been performed on this cell line. For each experiment, two 384-well plates were used allowing working with duplicates for combination rays and with quadruplicates for single agent rays.

Inhibition of cell growth was estimated after treatment with single compounds or combination of compounds for four days and comparing the signal to cells treated with vehicle (DMSO) and following equation described in example 1.

These measurements allow determining the potential synergistic combinations in using the statistical method described in example 1.

Results of In Vitro Studies

Compound (I), as single agent, inhibited the proliferation of A2058 cells with an 1040 of 11,500 nM. Compound (II), as single agent, inhibited the proliferation of A2058 cells with an IC40 of 1,890 nM (see table 5 below).

TABLE 5
Absolute IC₄₀ estimations for
each compound alone in example 3
Absolute IC₄₀ of single agents are esti-
mated with a 4-parameter logistic model

| | Absolute IC₄₀ (nM) | |
|---|---|---|
| Compound (I) | 11,500 | [7,720; 17,300] |
| Compound (II) | 1,890 | [1,520; 2,350] |

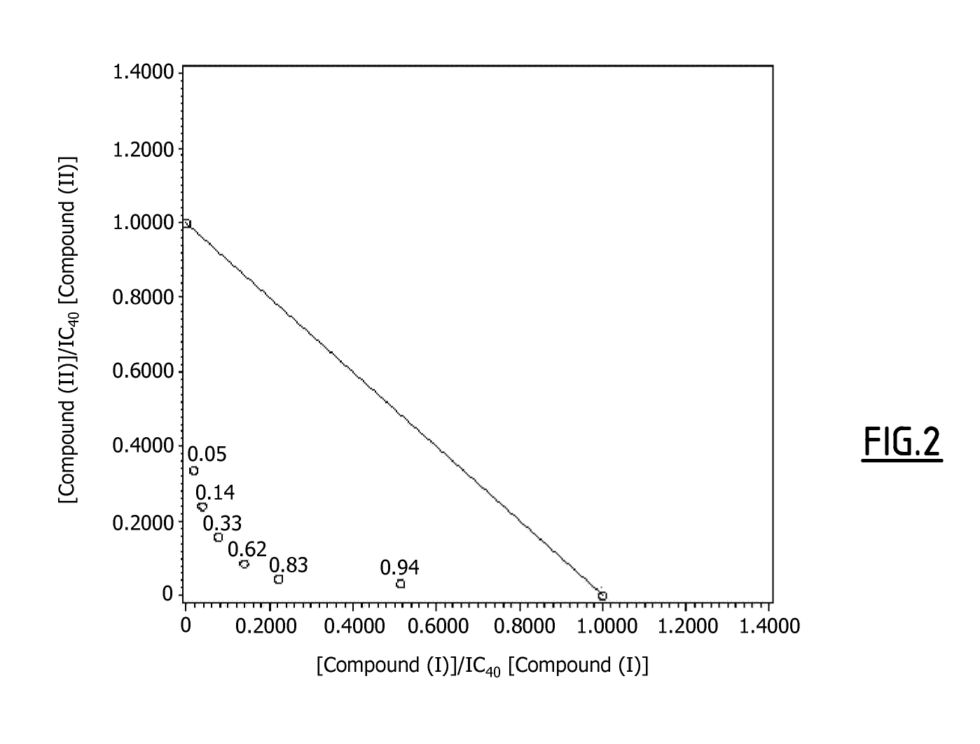

From the isobologram representation (FIG. 2) and the Table 6, significant synergy is observed with a Ki ranging from 0.23 to 0.55 for all effective fractions f of compound (I) in the mixture between 0.05 and 0.94.

TABLE 6
Interaction characterization in example 3
Interaction indexes (Ki) allow us to define the
interaction observed between the two compounds.

| | | Ki (confidence | | Interaction |
|---|---|---|---|---|
| | f values | interval at 95%) | | characterization |
| Ray 7 | 0.05 | 0.3549 | [0.2456; 0.5128] | Synergy |
| Ray 8 | 0.14 | 0.2795 | [0.1958; 0.3991] | Synergy |
| Ray 9 | 0.33 | 0.2351 | [0.164; 0.3371] | Synergy |
| Ray 10 | 0.62 | 0.2257 | [0.1497; 0.3404] | Synergy |
| Ray 11 | 0.83 | 0.268 | [0.1668; 0.4306] | Synergy |
| Ray 12 | 0.94 | 0.5474 | [0.3245; 0.9232] | Synergy |

These data correspond to a representative study out of 4 independent experiments.

For these four experiments, significant synergy or additivity with tendency to synergy was observed for all the effective fractions f of compound (I) in the mixture between 0.05 and 0.94.

### Example 4

### In Vitro Pro-Apoptotic Activity of Compound (I) in Combination with Compound (II) in Human Melanoma Cell Line A2058

To evaluate the pro-apoptotic activity of the PI3Kβ selective inhibitor compound (I) in combination with the RAF inhibitor compound (II), experiments were conducted using the human melanoma cell line A2058 (BRAF mutant, PTEN-deficient and unsensitive to said BRAF inhibitor). The characterization of the interaction between compound (I) and compound (II) was studied using western blotting method which allows investigating apoptosis by detecting the cleavage of the PARP protein.

Material and Methods

The human melanoma A2058 cell line was purchased at ATCC (Ref number CRL-11147 Batch 5074651). The A2058 cells were cultured in DMEM High glucose medium supplemented with 10% FBS and 2 mM L-Glutamine.

Compound (I) and compound (II) were prepared according to the material and methods of example 1.

Cell treatment, Western Blot experiment and calculation of apoptosis fold induction were performed according to the material and method described in example 2.

Results of In Vitro Studies

As compared to untreated cells, compound (I) or compound (II) as single agent at 10,000 nM concentration did not induce notable A2058 cell apoptosis with a fold induction of PARP cleavage of 1.17 and 1.00, respectively. In combination arm where the cells were treated with 10,000 nM of compound (I) and 10,000 nM of compound (II), the combined treatment induced A2058 cell apoptosis with a fold induction of PARP cleavage of 1.44. In the combination arm where the cells were treated with 10,000 nM of compound (I) and 1,000 nM of compound (II), the combined treatment induced A2058 cell apoptosis with an apoptosis fold induction of 1.61. Taxotere is shown as a positive control of apoptosis induction with a fold induction of PARP cleavage of 1.66 (see table 7).

These data correspond to a representative study out of 2 independent experiments.

GAPDH expression has been controlled in the lower panel of the western blots as a loading control.

TABLE 7
A2058 cell apoptosis induction for each compound
alone or in combination in example 4

| Concentration | PARP cleavage | Fold |
|---|---|---|
| (nM) | signal (AU-BG) | induction |
| DMSO 0.1% | 2146049 | 1.00 |
| Compound (I) 10 000 nM | 2141626 | 1.00 |
| Compound (II) 10 000 nM | 2501364 | 1.17 |
| Compound (I) 10 000 nM | 3087355 | 1.44 |
| Compound (II) 10 000 nM | | |
| Compound (I) 10 000 nM | 3448003 | 1.61 |
| Compound (II) 1 000 nM | | |
| Taxotere 100 nM | 3563893 | 1.66 |

### Example 5

### In Vitro Pro-Apoptotic Activity of Compound (I) in Combination with Compound (II) in Human Melanoma Cell Line WM-266-4 in a Concentration-Dependent Manner

To evaluate the pro-apoptotic activity of the PI3Kβ selective inhibitor compound (I) in combination with the RAF inhibitor compound (II), experiments were conducted using the human melanoma cell line WM-266-4 (BRAF mutant, PTEN-deficient, sensitive to BRAF inhibitor). The characterization of the interaction between compound (I) and compound (II) was studied using western blotting method which allows investigating apoptosis by detecting the cleavage of the PARP protein.

Material and Methods

The human melanoma WM-266-4 cell line was purchased at ATCC (Ref number CRL-1676 Batch 3272826). The WM-266-4 cells were cultured in RPMI1640 medium supplemented with 10% FBS and 2 mM L-Glutamine.

Compound (I) and compound (II) were dissolved in DMSO at concentration of 10 mM. They were diluted following two 10-fold step dilution in DMSO in order to obtain a 1 mM solution and a 0.1 mM solution Then each solution at 10 mM, 1 mM or 0.1 mM was diluted 50-fold in culture medium containing 10% serum before being added onto cells with a 20-fold dilution factor to reach final concentrations of 10,000 nM, 1,000 nM and 100 nM. The final DMSO concentration was 0.1% in controls and in all treated wells.

Cell treatment, Western Blot experiment and calculation of apoptosis fold induction were performed according to the material and method described in example 2.

Results of In Vitro Studies

As compared to untreated cells, compound (I) or compound (II) as single agent induce WM-266-4 cell apoptosis in a concentration-dependent manner with a fold induction of PARP cleavage of 6.86, 1.96, 0.94 (compound I) and 3.42, 2.59, 1.62 (compound II), at 10,000, 1,000 and 100 nM concentrations, respectively.

In combination arm where the cells were treated with increasing concentrations of compound (I) and 10,000 nM of compound (II), the combined treatment induced WM-266-4 cell apoptosis with a fold induction of PARP cleavage of 31.06, 11.69 and 4.69 at 10,000 nM, 1,000 nM and 100 nM compound (I) concentrations, respectively.

In combination arm where the cells were treated with increasing concentrations of compound (I) and 1,000 nM of compound (II), the combined treatment induced WM-266-4 cell apoptosis with a fold induction of PARP cleavage of 44.37, 12.56 and 3.76 at 10,000 nM, 1,000 nM and 100 nM compound (I) concentrations, respectively.

In combination arm where the cells were treated with increasing concentrations of compound (I) and 100 nM of compound (II), the combined treatment induced WM-266-4 cell apoptosis with a fold induction of PARP cleavage of 19.68, 3.84 and 1.65 at 10,000 nM, 1,000 nM and 100 nM compound (I) concentrations, respectively.

Overall, these data show that for all evaluated fractions of compounds (I) and (II), better apoptosis induction was observed for combined treatment as compared to single agents.

Taxotere is shown as a positive control of apoptosis induction with a fold induction of PARP cleavage of 1.56 (see table 8).

These data correspond to a representative study out of 2 independent experiments.

GAPDH expression has been controlled in the lower panel of the western blots as a loading control.

TABLE 8
WM-266-4 cell apoptosis induction for each compound
alone or in combination in example 5

| Concentration | PARP cleavage | Fold |
|---|---|---|
| (nM) | signal (AU-BG) | induction |
| DMSO 0.1% | 255858 | 1 |
| Compound (I) 10 000 nM | 1756263 | 6.86 |
| Compound (I) 1 000 nM | 502317 | 1.96 |
| Compound (I) 100 nM | 241428 | 0.94 |
| Compound (II) 10 000 nM | 876269 | 3.42 |
| Compound (II) 1 000 nM | 662217 | 2.59 |
| Compound (II) 100 nM | 413818 | 1.62 |
| Compound (II) 10 000 nM | 7948445 | 31.06 |
| Compound (I) 10 000 nM | | |
| Compound (II) 10 000 nM | 2991346 | 11.69 |
| Compound (I) 1 000 nM | | |
| Compound (II) 10 000 nM | 1200002 | 4.69 |
| Compound (I) 100 nM | | |
| Compound (II) 1 000 nM | 11353952 | 44.37 |
| Compound (I) 10 000 nM | | |
| Compound (II) 1000 nM | 3214871 | 12.56 |
| Compound (I) 1 000 nM | | |
| Compound (II) 1 000 nM | 961785 | 3.76 |
| Compound (I) 100 nM | | |
| Compound (II) 100 nM | 5034735 | 19.68 |
| Compound (I) 10 000 nM | | |
| Compound (II) 100 nM | 983014 | 3.84 |
| Compound (I) 1 000 nM | | |
| Compound (II) 100 nM | 421524 | 1.65 |
| Compound (I) 100 nM | | |
| Taxotere 100 nM | 399272 | 1.56 |

### Example 6

### In Vitro Pro-Apoptotic Activity of Compound (I) in Combination with Compound (II) in Human Melanoma Cell Line A2058 in a Concentration-Dependent Manner

To evaluate the pro-apoptotic activity of the PI3Kβ selective inhibitor compound (I) in combination with the RAF inhibitor compound (II), experiments were conducted using the human melanoma cell line A2058 (BRAF mutant, PTEN-deficient, unsensitive to BRAF inhibitor). The characterization of the interaction between compound (I) and compound (II) was studied using western blotting method which allows investigating apoptosis by detecting the cleavage of the PARP protein.

Material and Methods

The human melanoma A2058 cell line was purchased at ATCC (Ref number CRL-11147 Batch 5074651). The A2058 cells were cultured in DMEM High glucose medium supplemented with 10% FBS and 2 mM L-Glutamine.

Compound (I) and compound (II) were prepared according to the material and methods of example 5.

Cell treatment, Western Blot experiment and calculation of apoptosis fold induction were performed according to the material and method described in example 2.

Results of In Vitro Studies

As compared to untreated cells, compound (I) or compound (II) as single agent induce A2058 cell apoptosis in a concentration-dependent manner with a fold induction of PARP cleavage of 2.43, 1.26, 1.12 (compound I) and 2.44, 2.13, 1.14 (compound II), at 10,000, 1,000 and 100 nM concentrations, respectively.

In combination arm where the cells were treated with increasing concentrations of compound (I) and 10,000 nM of compound (II), the combined treatment induced A2058 cell apoptosis with a fold induction of PARP cleavage of 4.42, 4.05 and 2.97 at 10,000 nM, 1,000 nM and 100 nM compound (I) concentrations, respectively.

In combination arm where the cells were treated with increasing concentrations of compound (I) and 1,000 nM of compound (II), the combined treatment induced A2058 cell apoptosis with a fold induction of PARP cleavage of 6.73, 4.41 and 2.86 at 10,000 nM, 1,000 nM and 100 nM compound (I) concentrations, respectively.

In combination arm where the cells were treated with increasing concentrations of compound (I) and 100 nM of compound (II), the combined treatment induced A2058 cell apoptosis with a fold induction of PARP cleavage of 3.83, 2.04 and 1.28 at 10,000 nM, 1,000 nM and 100 nM compound (I) concentrations, respectively.

Overall, these data show that for all evaluated fractions of compounds (I) and (II), better apoptosis induction was observed for combined treatment as compared to single agents.

Taxotere is shown as a positive control of apoptosis induction with a fold induction of PARP cleavage of 6.79 (see table 9).

These data correspond to a representative study out of 2 independent experiments.

GAPDH expression has been controlled in the lower panel of the western blots as a loading control.

TABLE 9
A2058 cell apoptosis induction for each compound
alone or in combination in example 6

| Concentration | PARP cleavage | Fold |
|---|---|---|
| (nM) | signal (AU-BG) | induction |
| DMSO 0.1% | 2129347 | 1 |
| Compound (I) 10 000 nM | 5173100 | 2.43 |
| Compound (I) 1 000 nM | 2685395 | 1.26 |
| Compound (I) 100 nM | 2380877 | 1.12 |
| Compound (II) 10 000 nM | 5192876 | 2.44 |
| Compound (II) 1 000 nM | 4540797 | 2.13 |
| Compound (II) 100 nM | 2431268 | 1.14 |
| Compound (II) 10 000 nM | 9406114 | 4.42 |
| Compound (I) 10 000 nM | | |
| Compound (II) 10 000 nM | 8632233 | 4.05 |
| Compound (I) 1 000 nM | | |
| Compound (II) 10 000 nM | 6315320 | 2.97 |
| Compound (I) 100 nM | | |
| Compound (II) 1 000 nM | 14328382 | 6.73 |
| Compound (I) 10 000 nM | | |
| Compound (II) 1 000 nM | 9395466 | 4.41 |
| Compound (I) 1 000 nM | | |
| Compound (II) 1 000 nM | 6091204 | 2.86 |
| Compound (I) 100 nM | | |
| Compound (II) 100 nM | 8150664 | 3.83 |
| Compound (I) 10 000 nM | | |
| Compound (II) 100 nM | 4352371 | 2.04 |
| Compound (I) 1 000 nM | | |
| Compound (II) 100 nM | 2728777 | 1.28 |
| Compound (I) 100 nM | | |
| Taxotere 100 nM | 14448019 | 6.79 |

### Example 7

### In Vitro S6 Phosphorylation Inhibition of Compound (I) in Combination With Compound (II) in Human Melanoma Cell Line WM-266-4

To evaluate the inhibition of S6 phosphorylation by the PI3Kβ selective inhibitor compound (I) in combination with the BRAF inhibitor compound (II), experiments were conducted using the human melanoma cell line WM-266-4 (BRAF mutant and PTEN-deficient). The characterization of the interaction between compound (I) and compound (II) was studied using western blotting method measuring the expression of pS6.

Material and Methods

The human melanoma WM-266-4 cell line was purchased at ATCC (Ref number CRL-1676 Batch 3272826). The WM-266-4 cells were cultured in RPMI1640 medium supplemented with 10% FBS and 2 mM L-Glutamine.

Compound (I) and compound (II) were prepared according to the material and methods of example 5.

WM-266-4 cells were seeded into 6-well microplates at 1 000 000 cells per well, in complete culture medium and incubated at 37° C., 5% CO₂, overnight. Then, the cells were incubated in the presence or absence of compound (I) and in the presence or absence of compound (II) for 24 hours at 37° C. in the presence of 5% of CO₂.

At the end of cell treatment period, adherent cells as well as cells in the cell culture supernatant were lysed for the preparation of the proteins. Cells were lysed in a lysis buffer containing Hepes 50 mM, NaCl 150 mM, Glycerol 10%, Triton 1%, pH=7.5, adding extemporaneously a cocktail of protease and phosphatase inhibitors diluted 100 fold. Protein concentrations in each sample were determined using microBCA technique according to manufacturer's instructions. Western blotting was performed loading 20 μg of proteins in each gel well, and according to the operating procedure. pS6 was revealed using pS6 rabbit polyclonal antibody detecting phosphorylated S6 ribosomal protein (Ser240/244) followed by an anti-rabbit IgG HRP conjugate antibody. GAPDH was revealed in control using anti-GAPDH rabbit monoclonal antibody 14C10 followed by an anti-rabbit IgG HRP conjugate antibody. After western blotting revelation according to the operating procedure instructions, luminescence was read using FujiFilm (Ray Test) apparatus.

This instrument measures the total signal of luminescence obtained on Fujifilm machine (AU) for each selected band. Then, it subtracts the background value (BG) proportional to the size of the selected band or Area. The background is calculated from a band taken on the specific background of the western blot, to obtain the specific signal or (AU−BG) for each band. The calculation of the percentage of inhibition is performed for each treatment with compound or combination of compounds using the following formula:

Inhibition %=1−(((AU−BG)t/(AU−BG)st))*100

Where the values are defined as:

(AU−BG)t=Value of wells containing cells treated with the compounds (I) or (II) alone or in combination

(AU−BG)st=value of wells containing cells treated with the solvent (DMSO).

Results of In Vitro Studies

As compared to untreated cells, compound (I) or compound (II) as single agent significantly inhibited S6 phosphorylation in WM-266-4 in a concentration-dependent manner with inhibition percentages of 94, 90 and 69 (compound I) or 94, 92 and 76 (compound II), at 10,000, 1,000 and 100 nM concentrations, respectively.

In combination arm where the cells were treated with increasing concentrations of compound (I) and 10,000 nM of compound (II), the combined treatment significantly inhibited S6 phosphorylationin WM-266-4 with inhibition percentages of 97, 97 and 96 at 10,000, 1,000 and 100 nM compound (I) concentrations, respectively.

In combination arm where the cells were treated with increasing concentrations of compound (I) and 1,000 nM of compound (II), the combined treatment inhibited significantly S6 phosphorylation in WM-266-4 with inhibition percentages of 96, 96 and 95 at 10,000, 1,000 and 100 nM compound (I) concentrations, respectively.

In combination arm where the cells were treated with increasing concentrations of compound (I) and 100 nM of compound (II), the combined treatment inhibited significantly S6 phosphorylation in WM-266-4 with inhibition percentages of 97, 94 and 88 at 10,000, 1,000 and 100 nM compound (I) concentrations, respectively (see table 10).

Overall, these data show that combined treatments allowed sustained (>88% inhibition) pS6 inhibition even for the lowest concentrations as compared to single agents.

These data correspond to a representative study out of 2 independent experiments.

GAPDH expression has been controlled in the lower panel of the western blots as a loading control.

TABLE 10
Inhibition of S6 phosphorylation in WM-266-4 cell
each compound alone or in combination in example 7

| Concentration | pS6 signal | |
|---|---|---|
| (nM) | (AU-BG) | Inhibition % |
| DMSO 0.1% | 38859476 | 0 |
| Compound (I) 10 000 nM | 2239754 | 94 |
| Compound (1) 1 000 nM | 3984365 | 90 |
| Compound (I) 100 nM | 12132104 | 69 |
| Compound (II) 10 000 nM | 2333062 | 94 |
| Compound (II) 1 000 nM | 3163194 | 92 |
| Compound (II) 100 nM | 9230994 | 76 |
| Compound (II) 10 000 nM | 1202022 | 97 |
| Compound (I) 10 000 nM | | |
| Compound (II) 10 000 nM | 1276788 | 97 |
| Compound (I) 1 000 nM | | |
| Compound (II) 10 000 nM | 1630013 | 96 |
| Compound (I) 100 nM | | |
| Compound (II) 1 000 nM | 1521574 | 96 |
| Compound (I) 10 000 nM | | |
| Compound (II) 1 000 nM | 1474504 | 96 |
| Compound (I) 1 000 nM | | |
| Compound (II) 1 000 nM | 2017352 | 95 |
| Compound (I) 100 nM | | |
| Compound (II) 100 nM | 1208356 | 97 |
| Compound (I) 10 000 nM | | |
| Compound (II) 100 nM | 2156140 | 94 |
| Compound (I) 1 000 nM | | |
| Compound (II) 100 nM | 4799658 | 88 |
| Compound (I) 100 nM | | |

### Example 8

### In Vitro S6 Phosphorylation Inhibition of Compound (I) in Combination With Compound (II) in Human Melanoma Cell Line A2058

To evaluate the inhibition of S6 phosphorylation by the PI3Kβ selective inhibitor compound (I) in combination with the BRAF inhibitor compound (II), experiments were conducted using the human melanoma cell line A2058 (BRAF mutant and PTEN-deficient). The characterization of the interaction between compound (I) and compound (II) was studied using western blotting method measuring the expression of pS6.

Material and Methods

The human melanoma A2058 cell line was purchased at ATCC (Ref number CRL-11147 Batch 5074651). The A2058 cells were cultured in DMEM High glucose medium supplemented with 10% FBS and 2 mM L-Glutamine.

Compound (I) and compound (II) were prepared according to the material and methods of example 5.

Cell treatment, Western Blot experiment and calculation of percentage of pS6 inhibition were performed according to the material and method described in example 7.

Results of In Vitro Studies

As compared to untreated cells, compound (I) or compound (II) as single agent significantly inhibited S6 phosphorylation in A2058 in a concentration-dependent manner with inhibition percentages of 73, 42 and 8 (compound I) or 62, 40 and 27 (compound II), at 10,000, 1,000 and 100 nM concentrations, respectively.

In combination arm where the cells were treated with increasing concentrations of compound (I) and 10,000 nM of compound (II), the combined treatment significantly inhibited S6 phosphorylation in A2058 with inhibition percentages of 87, 60 and 63 at 10,000, 1,000 and 100 nM compound (I) concentrations, respectively.

In combination arm where the cells were treated with increasing concentrations of compound (I) and 1,000 nM of compound (II), the combined treatment inhibited significantly S6 phosphorylation in A2058 with inhibition percentages of 86, 65 and 59 at 10,000, 1,000 and 100 nM compound (I) concentrations, respectively.

In combination arm where the cells were treated with increasing concentrations of compound (I) and 100 nM of compound (II), the combined treatment inhibited significantly S6 phosphorylation in A2058 with inhibition percentages of 86, 69 and 34 at 10,000, 1,000 and 100 nM compound (I) concentrations, respectively (see table 11).

Overall, these data show that combined treatments allowed increased pS6 inhibition for the tested concentrations as compared to single agents.

These data correspond to a representative study out of 2 independent experiments.

GAPDH expression has been controlled in the lower panel of the western blots as a loading control.

TABLE 11
Inhibition of S6 phosphorylation in A2058 cell each
compound alone or in combination in example 8

| Concentration | pS6 signal | |
|---|---|---|
| (nM) | (AU-BG) | Inhibition % |
| DMSO 0.1% | 29871720 | 0 |
| Compound (I) 10 000 nM | 8161752 | 73 |
| Compound (I) 1 000 nM | 17416427 | 42 |
| Compound (I) 100 nM | 27394341 | 8 |
| Compound (II) 10 000 nM | 11383792 | 62 |
| Compound (II) 1 000 nM | 17943024 | 40 |
| Compound (II) 100 nM | 21830955 | 27 |
| Compound (II) 10 000 nM | 3957100 | 87 |
| Compound (I) 10 000 nM | | |
| Compound (II) 10 000 nM | 11963944 | 60 |
| Compound (I) 1 000 nM | | |
| Compound (II) 10 000 nM | 11172660 | 63 |
| Compound (I) 100 nM | | |
| Compound (II) 1 000 nM | 4243930 | 86 |
| Compound (I) 10 000 nM | | |
| Compound (II) 1000 nM | 10310189 | 65 |
| Compound (I) 1 000 nM | | |
| Compound (II) 1 000 nM | 12233256 | 59 |
| Compound (I) 100 nM | | |
| Compound (II) 100 nM | 4159884 | 86 |
| Compound (I) 10 000 nM | | |
| Compound (II) 100 nM | 9180942 | 69 |
| Compound (I) 1 000 nM | | |
| Compound (II) 100 nM | 19754830 | 34 |
| Compound (I) 100 nM | | |

### Summary of In Vitro Results

### Examples 1 to 8

By the above data, it is demonstrated that a selective PI3Kβ inhibitor (compound I) can synergize with a BRAF inhibitor as vemurafenib (compound II) to increase the inhibitory activity on cell proliferation and the induction of cell death in melanoma cells responsive (here WM-266-4 cell line) or unsensitive (here A2058 cell line) to BRAF inhibitor (here vemurafenib) and exhibiting PI3K pathway activation through PTEN deficiency and MAPK pathway activation, in particular through BRAF activating mutations.

FIGS. 1 and 2: Isobologram Representation of Example 1 and 3: In Vitro Anti-Proliferative Activity of Compound (I) in Combination with Compound (II) in Human Melanoma Cell Line WM-266.4 and A2058

The isobologram representation permits to visualize the position of each ray according to the additivity situation represented by the line joining the point (0,1) to the point (1,0). All rays below this line correspond to a potential synergistic situation whereas all rays above the line correspond to a potential antagonistic situation.

For example 1 experiment, according to the isobologram representation, rays with an effective fraction f between 0.05 and 0.62 are below the additivity line with significant synergy for all rays (see FIG. 1). For example 3 experiment, according to the isobologram representation, rays with a effective fraction f between 0.05 and 0.94 are below the additivity line with significant synergy for all rays (see FIG. 2).

1. A method of treatment of a patient having resistant cancer cells to at least one RAF inhibitor, comprising administering a combination of a PI3Kβ inhibitor with a RAF inhibitor to said patient.

2. The method according to claim 1, wherein the resistance to the at least one RAF inhibitor is the resistance to said RAF inhibitor of the combination.

3. The method according to claim 1, wherein the patient suffers from melanoma.

4. The method according to claim 1, wherein the melanoma cells have an activating BRAF mutation.

5. The method according to claim 1, wherein the melanoma cells are PTEN deficient.

6. The method according to claim 1, wherein the PI3Kβ inhibitor is of formula (I): 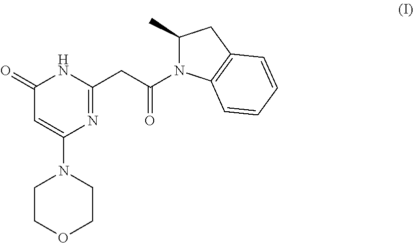 [SMILES: C[C@H]1Cc2ccccc2N1C(=O)Cc1nc(N2CCOCC2)cc(=O)[nH]1]
or one of its pharmaceutically acceptable salts thereof.

7. The method according to claim 1, wherein the RAF inhibitor is of formula (II): 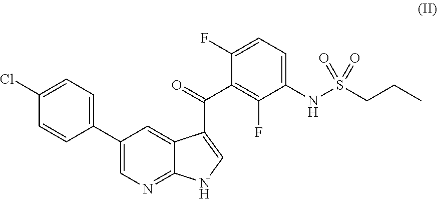 [SMILES: CCCS(=O)(=O)Nc1ccc(F)c(C(=O)c2c[nH]c3ncc(-c4ccc(Cl)cc4)cc23)c1F]
or one of its pharmaceutically acceptable salts thereof.

8. The method according to claim 1, wherein said combination inhibits tumor cell growth.

9. The method according to claim 1, wherein the administration of the PI3Kβ inhibitor and the RAF inhibitor is a simultaneous, a separate or a sequential administration.

10. The method according to claim 9, wherein the administration is separate or sequential and wherein the administration of the PI3Kβ inhibitor is followed by the administration of the RAF inhibitor.

11. The method according to claim 9, wherein the administration is separate or sequential and wherein the administration of the RAF inhibitor is followed by the administration of the PI3Kβ inhibitor.

12. A method of determining the efficiency of inhibition of tumor cell growth by a combination of a PI3Kβ inhibitor with a RAF inhibitor, wherein said tumor cells are cancer cells resistant to at least one RAF inhibitor, comprising determining the amount of phosphorylated ribosomal protein S6 (pS6) in said cancer cells resistant to at least one RAF inhibitor.

13. An in vitro method of monitoring the response of a patient, having cancer cells resistant to at least one RAF inhibitor, to a combination of a PI3Kβ inhibitor with a RAF inhibitor, said method comprising:
i) determining the amount of protein pS6 in cancer cells resistant to at least one RAF inhibitor of said patient at a first time point,
ii) determining the amount of protein pS6 in cancer cells resistant to at least one RAF inhibitor of said patient at a later time point,
iii) comparing the amount of protein pS6 of step i) with the amount of protein pS6 in step ii), and
iv) determining that the patient responds to said combination if the amount of protein pS6 of step i) is equal or superior to the amount of protein pS6 in step ii).

14. The method according to claim 13, wherein the resistance to the at least one RAF inhibitor is the resistance to said RAF inhibitor of the combination.

15. The method according to claim 1, wherein the PI3Kβ inhibitor is of formula (I): 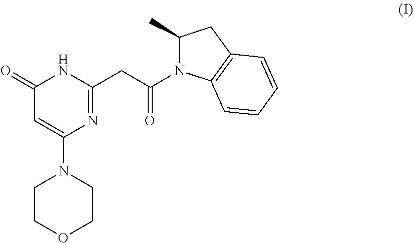 [SMILES: C[C@H]1Cc2ccccc2N1C(=O)Cc1nc(N2CCOCC2)cc(=O)[nH]1]
or one of its pharmaceutically acceptable salts thereof, and
the RAF inhibitor is of formula (II): 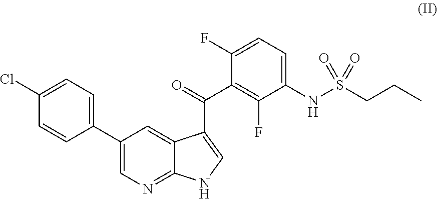 [SMILES: CCCS(=O)(=O)Nc1ccc(F)c(C(=O)c2c[nH]c3ncc(-c4ccc(Cl)cc4)cc23)c1F]
or one of its pharmaceutically acceptable salts thereof.

16. The method according to claim 4, wherein the activating BRAF mutation is a BRAF-V600E mutation or a BRAF-V600K mutation.
